(19) United States
(12) Patent Application Publication
(10) Pub. No.: US 2004/0092511 A1
(43) Pub. Date: May 13, 2004

(54) Pharmaceutical combinations and their use in treating gastrointestinal and abdominal viscera disorders

(76) Inventors: Stephan Anthony Billstein, Franklin Lakes, NJ (US); Peter Dumovic, Mendham, NJ (US); Nicola Franco, Sondersdorf (FR); Mark Thomas Iwicki, Long Valley, NJ (US); Hans-Jurgen Pfannkuche, Weil am Rhein (DE); Edward Joseph Wilusz JR., Pittstown, NJ (US)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/702,688

(22) Filed: Nov. 6, 2003

Related U.S. Application Data
(63) Continuation of application No. 09/722,784, filed on Nov. 27, 2000, now abandoned.
(60) Provisional application No. 60/266,333, filed on Dec. 10, 1999.

(51) Int. Cl. A61K 31/5513; A61K 31/445

(52) U.S. Cl. 514/221; 514/282; 514/317

(57) ABSTRACT

The present invention relates to a combination of a first agent including either a 5-HT₄ receptor agonist or antagonist or a 5-HT₃ receptor antagonist and a co-agent and pharmaceutical compositions and formulations containing the combination. The present invention also relates to a method of treating a gastrointestinal and abdominal viscera disorder by administering the pharmaceutical compositions to a patient. The pharmaceutical compositions may also be employed as laxatives, to prepare a patient for colonoscopy and to regulate and stabilize enterochromaffin cell secretory, pain and motility mechanisms, afferent fiber activity and GI and lower abdominal smooth muscle cells. The dosage is preferably oral and administration is preferably once or twice a day. The preferred first agent is tegaserod.

### BACKGROUND

Pharmaceutical compounds have been employed to act as 5-HT₄ agonists or antagonists and/or 5-HT₃ antagonists in mammals. As serotonin 5-HT₄ agonists, these compounds are gastrointestinal motility agents useful for the treatment of mammalian gastrointestinal (GI) motility disorders such as reflux esophagitis, gastroparesis, non-ulcer dyspepsia, ileus, constipation and irritable or inflammatory bowel syndrome (“IBS”). As serotonin 5-HT₄ antagonists these compounds are useful in the treatment of motility disorders of the GI tract such as diarrhea and diarrhea-predominant irritable bowel syndrome. As serotonin 5-HT₃ antagonists these compounds are useful in the treatment of diarrhea and diarrhea-predominant irritable bowel syndrome. The serotonin 5-HT₄ agonists or antagonists and/or serotonin 5-HT₃ antagonists are also useful in the treatment of emesis, anxiety, visceral pain, substance abuse (either cravings or withdrawal syndrome), cognitive disorders and other CNS disorders wherein treatment with a serotonin 5-HT₄ agonist or antagonist and/or serotonin 5-HT₃ antagonist would be indicated.

Serotonin (5-hydroxytryptamine; 5-HT) functions as a neurotransmitter in the mammalian central nervous system (CNS) and in the periphery. Serotonin is one of the transmitters to be recognized for its physiological importance, and agents which interact with 5-HT receptors are currently the focus of much research. P. Bonate, Clinical Neuropharmacology, Vol. 14(1), pp. 1-16 (1991). Serotonin is unsurpassed among monoamine neurotransmitters in the number of receptor subtypes identified. To date, the number of subtypes is into the teens, including the major classes, i.e., 5-HT1, 5-HT2, 5-HT3, 5-HT4, 5-HT5, 5-HT6, and 5-HT7. Because of the multiplicity of serotonin receptor sybtypes, the identification of which serotonin receptor subtype is correlated to various physiological/pharmacological actions is complicated.

Serotonin has been known for some years to modulate peristalsis in the GI tract in various mammalian models. During the mid 1980s, several specific antagonists to the 5-HT₃ receptor subtype were identified from independent laboratories. 5-HT₃ receptor antagonists are currently used as anti-emesis/vomiting agents in cancer therapy. 5-HT₃ antagonists have also recently been studied for the treatment of IBS.

A number of gastrointestinal syndromes are related to the production and actions of serotonin, and they have a fairly common occurrence in a very large number of people in the western world. Some of the more well-known gastrointestinal conditions, syndromes or diseases are IBS, gastro-esophageal reflux disease (“GERD”) and dyspepsia.

IBS is a chronic condition associated with abdominal pain, bloating and altered bowel function and is estimated to affect as much as 10-20% of the population. It is associated equally with women and men. The diagnosis is made by exclusion, by ruling out structural and/or biochemical abnormalities. Sometimes the disease is referred to as irritable colon, spastic colon, spastic colitis or mucous colitis. The latter two are almost certainly misnomers, as colitis implies inflammation of the colon, and an absence of inflammation is one of the defining observations in a diagnosis of IBS. IBS is one of the most common diagnoses made in patients referred to gastroenterologists. It represents half of GP referrals to a gastroenterologist, according to data from the Center for Current Research Inc. The cause of IBS is unknown, but a number of factors have been implicated, including diet, lifestyle, depression, anxiety, infection and unrelated inflammatory conditions, including early insult resulting in central sensitization and sensitizing of neurons in the gut. Many physicians' believe that there is a psychological element to the disease. Many currently used and potential treatments for IBS have failed to establish a meaningful clinical impact.

Dyspepsia is also an important health problem. Up to 5% of all visits to family doctors are for this problem. When a doctor is consulted for a problem of dyspepsia, they will usually take a careful history and perform a physical examination. The most common conditions that are associated with patients who present with chronic symptoms of dyspepsia are GERD which occurs in up to 20% of patients, duodenal ulcer or gastric ulcer which occurs in up to 5% of patients and other diagnoses in up to 15% of patients (example of other diagnoses may be functional dyspepsia, non-ulcerative dyspepsia, gallbladder or liver disease). However, in a large proportion of patients (up to 60%) no clear cause (such as an ulcer) can be found.

Gastroesophageal reflux is a condition that is associated with the reflux of gastric contents to the esophagus through the lower esophageal sphincter. GERD is characterized by symptoms of heartburn, bloating, abdominal pain, epigastric pain, early satiety, nausea, regurgitation, burbulence and vomiting. The reflux is thought to occur because transient lower esophageal sphincter relaxations allow gastric contents to enter the esophagus. Conditions associated with abnormal esophageal sphincter function are pregnancy, GERD, hiatal hernia, obesity, recurrent or persistent vomiting, and nasogastric tubes. The risk factors of this condition can increase with previous esophageal surgery, or known esophageal stricture. The prevalence is 5 out of 1,000 people. Evidence indicates that up to 36% of otherwise healthy Americans suffer from heartburn at least once a month, and that 7% experience heartburn as often as once a day. The incidence of GERD increases markedly after the age of 40, and it is not uncommon for patients experiencing symptoms to wait years before seeking medical treatment. Nebel O. T. et al, Am. J. Dig. Dis., Vol. 21(11): 953-956 (1976); and Spechler S. J., et al., Digestion. Vol. 51(suppl. 1):24-29 (1992).

Current therapies for functional gastrointestinal disorders are either OTC or prescription products or a combination of both. These diseases are characterized by altered motility, sensitivity and secretion as well as having a psychological (usually subconscious) overlay as well. The presently prescribed medications lose their efficacy value. Various reasons for this loss of efficacy have been postulated. Some of them include a development of tolerance, intolerability of accompanying adverse effects, relief of the motility component but not the other symptoms and signs such as pain and bloating etc.

Accordingly, there is a need for agents which modulate and normalize GI altered motility, sensitivity and secretion and interact with 5-HT receptors and which have broad clinical usefulness for the treatment of the many gastrointestinal disorders which affect millions of people each year. More specifically, there is a need for pharmaceutical combinations comprising either 5-HT₄ receptor agonists or antagonists, 5-HT₄ receptor partial agonists or 5-HT₃ receptor antagonists and a co-agent effective for the treatment of gastrointestinal altered motility, sensitivity and secretion and abdominal viscera disorders including both functional and organic diseases. In a preferred embodiment the pharmaceutical combination comprises tegaserod (described below) and a co-agent.

There is also a need for pharmaceutical compositions comprising the pharmaceutical combination and a pharmaceutically acceptable carrier.

There is also a need for a method of treating a patient suffering from a gastrointestinal altered motility, sensitivity and secretion and abdominal viscera disorders comprising the administration of a therapeutically effective amount of the pharmaceutical combinations and compositions to the patient.

There is also a need for a method of regulating, stabilizing and normalizing gastrointestinal and abdominal viscera disorders comprising administering a therapeutically effective amount of the pharmaceutical combinations and compositions to a patient.

### SUMMARY

Toward these ends, and others, an aspect of the invention provides a pharmaceutical combination comprising a first agent and a co-agent for the treatment of gastrointestinal altered motility, sensitivity and secretion and abdominalviscera disorders. This combination may also be used to regulate, stabilize and normalize gastrointestinal altered motility, sensitivity and secretion abdominal viscera disorders.

The first agent includes 5-HT₄ receptor agonists or antagonists, 5-HT₄ receptor partial agonists and 5-HT₃ receptor antagonists. In a preferred embodiment the first agent is a 5-HT₄ receptor partial agonist, for example, the compounds of formula I disclosed in co-owned U.S. Pat. No. 5,510,353, which is incorporated herein in its entirety as if set forth in full herein. In an even more preferred embodiment the first agent is tegaserod which is a compound of formula

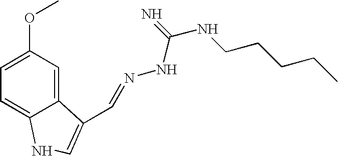

(herein “tegaserod”).

The co-agent is selected from the following groups of compounds: 5-HT₃ receptor antagonists, 5-HT₄ receptor agonists or antagonists, H₂ antagonists, proton pump inhibitors (“PPIs”) which include irreversible, reversible and pre PPIs, anxiolytics, benzodiazepine compounds, anti-spasmodic/anti-muscarinic agents, selective serotonin reuptake inhibitors (“SSRIs”), tricyclic antidepressants, selegeline, belladonna alkaloids, muscarinic, (“M₁”) antagonists, metoclopramide, cholecystokinin (“CCK”) receptor antagonists, kappa opioid agonists or antagonists, motilin receptor agonists or antagonists, nitric oxide synthase inhibitors, benzimidazolone derivatives, especially derivatives of endo-N-(8 methyl-8azabicyclo[3,2,1] oct-3yl-1H-3-benzimidazolone or AZB class derivatives (“collectively BIMU compounds”), GABA_{B} receptor agonists or modulators, Neurokinin (“NK”) receptor agonists or antagonists, substance P agonists or antagonists, calcitonin gene-related peptide receptor agonists or antagonists, endorphin/enkephalin analogs, anti-inflammatory compounds, stimulant laxatives, osmotic laxatives, fecal softeners, absorbents and fiber supplements, antacids, GI relaxants, loperamide, diphenoxylate, anti-gas compounds, bismuth-containing preparations, subsalicylate, pentosan polysulfate, hydroxyzine, dextromethorphans, anti-emetic dopamine D₂ antagonists and mast cell stabilizing agents.

In accordance with another aspect of the present invention there is provided a pharmaceutical composition comprising a combination of a first agent including 5-HT₄ receptor agonists or antagonists, 5-HT₄ receptor partial agonists and 5-HT₃ receptor antagonists, a co-agent and a pharmaceutically acceptable carrier. In a preferred embodiment the first agent is a 5-HT₄ receptor partial agonist, for example, the compound of formula I disclosed in co-owned U.S. Pat. No. 5,510,353. In an even more preferred embodiment the first agent is tegaserod.

In another embodiment of the present invention there is provided a method of treating a patient suffering from gastrointestinal altered motility, sensitivity and secretion and abdominal viscera disorders comprising administering a therapeutically effective amount of a pharmaceutical composition comprising the pharmaceutical combination of a first agent and a co-agent, including the pharmaceutically acceptable salts, racemates or enantiomers thereof, in the presence of a pharmaceutically acceptable carrier to the patient.

The gastrointestinal altered motility, sensitivity and secretion and abdominal viscera disorders which may be treated with the above-identified pharmaceutical combinations and compositions include heartburn, bloating, postoperative ileus, abdominal pain and discomfort, early satiety, epigastric pain, nausea, vomiting, burbulence, regurgitation, intestinal pseudoobstruction, anal incontinence, chronic constipation, diabetic gastroparesis, dyspepsia, GERD, IBS, ulcerative colitis, Crohn's disease, menstrual cramps, spastic and interstitial cystitis and ulcers and the visceral pain associated therewith.

The pharmaceutical combinations and compositions may also be employed as laxatives, as a preparation for a patient for colonoscopy, and as a means of regulating, stabilizing and normalizing gastrointestinal and abdominal viscera disorders, for example, regulating, stabilizing and normalizing enterochromaffin cell secretory, pain and motility mechanisms, afferent fiber activity and GI and lower abdominal smooth muscle cells.

Other objects, features, advantages and aspects of the present invention will become apparent to those of skill from the following description. It should be understood, however, that the following description and the specific examples, while indicating preferred embodiments of the invention, are given by way of illustration only. Various changes and modifications within the spirit and scope of the disclosed invention will become readily apparent to those skilled in the art from reading the following description and from reading the other parts of the present disclosure.

Definitions

Unless otherwise specified herein, common definitions are intended by the words and terms used herein. For example, the term “pharmaceutical combination” as used herein means a product that results from the mixing or combining of more than one active ingredient and includes both fixed and non-fixed combinations of the active ingredients.

The term “fixed combination” as that term is used herein means that the active ingredients, e.g. tegaserod and a co-agent, are both administered to a patient simultaneously in the form of a single entity or dosage. As an example, a fixed combination would be one capsule containing two active ingredients.

The term “non-fixed combination” as that term is used herein means that the active ingredients, e.g. tegaserod and a co-agent, are both administered to a patient as separate entities either simultaneously, concurrently or sequentially with no specific time limits, wherein such administration provides therapeutically effective levels of the two compounds in the body at the same time. As an example, a non-fixed combination would be two capsules each containing one active ingredient where the purpose is to have the patient achieve treatment with both active ingredients together in the body.

The term “therapeutically effective amount” shall mean that amount of drug or pharmaceutical agent that will elicit the biological or medical response of a tissue, system or animal (mammal) that is being sought by a researcher or clinician. A “therapeutically effective amount” can be administered in both a fixed or non-fixed combination of tegaserod and a co-agent.

The term “a gastrointestinal altered motility, sensitivity and secretion disorder(s)” as used herein includes one or more of the symptoms and conditions which affect the gastrointestinal tract from the mouth to the anus, which include, but are not limited to, heartburn, bloating, postoperative ileus, abdominal pain and discomfort, early satiety, epigastric pain, nausea, vomiting, burbulence, regurgitation, intestinal pseudoobstruction, anal incontinence, GERD, IBS, dyspepsia, chronic constipation, diabetic gastroparesis, ulcerative colitis, Crohn's disease, menstrual cramps, spastic and interstitial cystitis and ulcers and the visceral pain associated therewith.

The term “abdominal viscera disorder(s)” as used herein includes those condition which affect the smooth muscles of the lower abdomen outside of the GI tract and include but are not limited to those conditions treated by regulation, stabilization and normalization of enterochromaffin cell secretory, pain and motility mechanisms, afferent fiber actvity and GI and lower abdominal smooth muscle cells.

The term “gastro-esophageal reflux disease” and “GERD” as used herein means the incidence of, and the symptoms of, those conditions caused by the reflux of the stomach contents into the esophagus. This includes all forms/manifestations of GERD including, but not limited to, erosive and non-erosive GERD, heartburn and other symptoms associated with GERD.

The term “irritable bowel syndrome” and “IBS” as used herein means a disorder of function involving altered motility, sensitivity and secretion involving the small intestine and large bowel associated with variable degrees of abdominal pain, constipation, bloating or diarrhea without bowel inflammation.

The term “dyspepsia” as used herein means a condition characterized by symptoms of abdominal pin, epigastric pain, bloating, early satiety, nausea, heartburn and vomiting as a primary gastrointestinal dysfunction or as a complication due, and not exclusive to other disorders such as appendicitis, gallbladder disturbances, or malnutrition.

The term “gastroparesis” as used herein means a paralysis of the stomach brought about by a motor abnormality in the stomach which is often manifested as delayed gastric emptying. This can also be a complication of diseases such as diabetes, progressive systemic sclerosis, anorexia nervosa, or myotonic dystrophy.

The term “constipation” as used herein means a condition characterized by infrequent or difficult evacuation of feces resulting from conditions such altered GI motility, altered sensation or evacuation functions and altered reabsorption of water.

The terms “mammal”, “mammalian organism” or “patient” are used interchangeably herein and include, but are not limited to, humans, dogs, cats, horses and cows. The preferred patients are humans.

The term “treat” or “treatment” encompasses the complete range of therapeutically positive effects associated with pharmaceutical medication including reduction of, alleviation of and relief from the symptoms or illness which affect the organism.

### DESCRIPTION

In one aspect of the invention there is provided a pharmaceutical combination comprising a first agent and a co-agent for the treatment of a gastrointestinal altered motility, sensitivity and secretion and abdominal viscera disorder, for use as a laxative or to prepare a patient for colonoscopy and to regulate and stabilize gastrointestinal and abdominal viscera disorders. The first agent includes 5-HT₄ receptor agonists or antagonists, 5-HT₄ receptor partial agonists and 5-HT₃ receptor antagonists.

The 5-HT₄ receptor agonists and partial agonists include any compound which can activate 5-HT₄ receptors under quiescent/resting conditions (complete or partial activation). These compounds include, but are not limited to, the compounds of formula I disclosed in co-owned U.S. Pat. No. 5,510,353, tegaserod, cisapride, nor-cisapride, renzapride, zacopride, mosapride, prucalopride, SB 205149, SC 53116, RS 67333, RS 67506, BIMU 1, BIMU 8 and (S)-RS 56532. Cisapride, cis-4-amino-5-chloro-N-[1-[3-(4-fluorphenoxy)propyl]-3-methoxy-4-piperidinyl]-2-methoxybenzamide and nor-cisapride are used as gastro-prokinetic agents (See A. Reyntjens et al., Drug Div. Res., 8, 251 (1986) and Curr. Ther. Res., 36, 1029-1070 (1984)). The compound is marketed internationally under trade names such as ACENALIN®, PREPULSID®, RISAMOL®, PULSAR® and PROPULSIN®. Preferred first agents are 5-HT₄ receptor partial agonists, for example, tegaserod.

5-HT₄ receptor antagonists includes any compounds which bind to the 5-HT₄ receptor as defined by the IUPHAR (See Pharmacological Reviews, Vol. 44, p. 157-213 (1994) and that antagonizes the effect of serotonin. A relevant test to determine whether or not a compound is a 5-HT₄ receptor antagonist is the Guinea-Pig distal colon test as described in Br. J. Pharm., p. 1593-1599 (1993) or in the test described in Arch. Pharmacol., Vol. 343, p. 439-446 (1991). 5-HT₄ receptor antagonists include: piboserod; A-85380 (Abbott Laboratories) (WO 94/08994);SB 204070 (SmithKline Beecham) (Drugs Fut., 19:1109-1121 (1994));; SB 207058 (SmithKline Beecham) (Exp. Opin. Invest. Drugs, 3(7):767 (1994)); SB 207710 (SmithKline Beecham) (Drug Data Report, 15(10):949 (1993)); SB 205800 (SmithKline Beecham) (Drug Data Report, 15(10):949 (1993)); SB 203186 (SmithKline Beecham) (Br. J. Pharmacol., 110:10231030 (1993)); N 3389 (Nisshin Flour Milling) (Eur. J. Pharmacol., 271:159 (1994)); FK 1052 (Fujisawa) (J. Pharmacol. Exp. Ther., 265:752 (1993)); SC 56184 (Searle) (R&D Focus, 2(37) 10 (1993)); SC 53606 (Searle/Monsanto) (J. Pharmacol. Exp. Ther. 226:1339); DAU 6285 (Boerhinger Ingelheim) (Br. J. Pharmacol., 105:973 (1992)); GR 125487 (Glaxo) (Br. J. Pharmacol., 113 suppl. 119P & 120P (1994)); GR 113808 (Glaxo) (Br. J. Pharmacol. 110:1172); RS 23597 (Syntex) (Bioorg Med. Chem. Lett., 4(20):2477 (1994)); RS 39604 (Syntex) (Br. J. Pharmacol., 115, 1087-1095 (1995)); LY0353433 (Eli Lilly Co. Ltd.) (J. Pharmacol. Exp. Ther., 277(1), 97-104 (1996)); and R 59595 (Eur. J. Pharmacol., 212 (1992), 51-59).

5-HT₃ receptor antagonists include compounds which bind to the 5-HT₃ receptor and antagonize the effect of a 5-HT₃ receptor agonist, such as cilansetron, (R)-5,6,9,10-tetrahydro-10-[2-methylimidazol-1-yl)methyl]-4H-pyrido[3,2,1jk]-carbazol-11(8H)-one, which is described in EP 29761 and in U.S. Pat. No. 5,663,343; alosetron which is described in WO 99/17755 and U.S. Pat. Nos. 5,342,627 and 5,425,950; ramosetron; azasetron; ondansetron; granisetron; and tropisetron.

Expressly included herein are the pharmaceutically acceptable salts, racemates or enantiomers of the above-described compounds.

In a preferred embodiment, the first agent is the compounds of formula I disclosed in co-owned U.S. Pat. No. 5,510,353. These compounds are aminoguanidine compounds with the structure

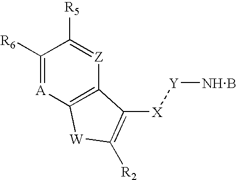

wherein

W is S or —NR₁— wherein R₁ is hydrogen, C₁₋₆alkyl or acyl,

R₂ is hydrogen, halogen or C₁₋₆alkyl,

R₅ is hydrogen; halogen; C₁₋₆alkyl; hydroxy; nitro; amino;

C₁₋₄alkylamino; acylamino; C₂₋₆alkoxycarbonyl; SO₂ NRₐR_{b} wherein each of Rₐ and R_{b} independently is hydrogen or C₁₋₆alkyl; cyano; or trimethylsilyl; or,

when A is —CR₇═, R₅ is also C₁₋₆alkyl substituted by —SO₂— C₁₋₆alkyl, —SO₂NRₐR_{b},

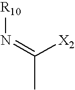

—CONRₐR_{b}, —NH—SO₂— C₁₋₆-alkyl, —N(C₁₋₆alkyl)-SO₂-(C₁₋₆alkyl), —NRₐR′_{b} wherein R′_{b} is hydrogen, C₁₋₆alkyl, C₃₋₇cycloalkyl, C₃₋₆alkenyl, phenyl or phenyl-C₁₋₃alkyl wherein the phenyl ring is optionally substituted, C₂₋₆alkoxycarbonyl, —PO(C₁₋₄alkyl)₂ or a heterocyclic radical; carboxy; CONRₐR_{b}; —PO(C₁₋₄alkyl)₂; OCONR_{c}R_{d}, wherein each of R_{c} and R_{d} independently is C₁₋₆alkyl; or a heterocyclic radical,

R₆ is hydrogen or, when R₅ is OH, R₆ is hydrogen or halogen,

Z is —CR4═ wherein R₄ is hydrogen, halogen, hydroxy or C₁₋₆alkyl or, when R₅ is hydrogen or hydroxy, Z is also —N═,

A is —N═ or —CR₇═ wherein R₇ is hydrogen, halogen, C₁₋₆alkyl or C₁₋₆ alkoxy, or R₁ and R₇ together represent —(CH₂)ₘ— or —X₃ (CH₂)ₚ—wherein m is 2, 3 or 4, p is 2 or 3 and X₃ is O, S or —N(C₁₋₆alkyl)-, X₃ being attached to the 6-membered ring,

X—Y is —CR₈═N— or —CH(R₈)—NH— wherein R₈ is hydrogen or C₁₋₆alkyl, and

B is a radical of formula (a) or (b),

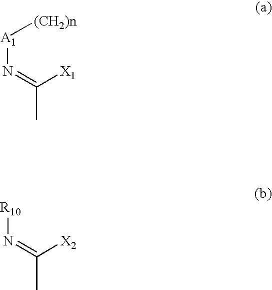

wherein n is 1 or 2,

A₁ is C═O or CH₂,

X₁ is S, NR₁₁ or CR₁₂R₁₃, wherein R₁₁ is hydrogen or acyl, each of R₁₂ and R₁₃ independently is hydrogen, C₁₋₄alkyl or C₅₋₇cycloalkyl,

R₁₀ is hydrogen; C₁₋₁₂alkyl; C₁₋₆alkyl substituted by hydroxy, aryl, aryloxy, adamantyl, a heterocyclic radical, —NR₁₅—CO—R₁₆ or —NH—SO₂-aryl; C₅₋₇cycloalkyl; aryl; adamantyl; acyl; or —CONHR₁₄,

wherein R₁₄ is C₁₋₁₀alkyl, C₅₋₇cycloalkyl, C₅₋₇cycloalkyl-C₁₋₄alkyl, aryl, arylC₁₋₄alkyl or a heterocyclic radical,

R₁₅ is hydrogen or C₁₋₄alkyl, and

R₁₆ is C₁₋₆alkyl, C₅₋₇cycloalkyl, C₅₋₇cycloalkyl-C₁₋₄alkyl, aryl or arylC₁₋₄alkyl, and

X₂ is —SR₂₀ or —NR₃R′₁₀ wherein R₂₀ is C₁₋₆alkyl, R₃ is hydrogen or C₁₋₆alkyl and R′₁₀ has one of the significances given for R₁₀ above, or R₃ and R′₁₀ together with the nitrogen atom to which they are attached form a 5-, 6- or 7-membered saturated, aromatic or non aromatically unsaturated heterocycle which may comprise a further heteroatom selected from N, S and O and which may be further condensed to a benzene ring, provided that

i) when B is a radical of formula (b), only one of R₁₀ and R′₁₀ can be other than hydrogen and X₂ can be —SR₂₀ only when R₁₀ is hydrogen, and

ii) R₅ is other than hydrogen when B is a radical

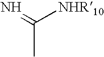

wherein R′₁₀ is 4-methylphenyl as aryl, each of Z and A is —CH═, W is —NH—, R₂ and R₆ are each hydrogen and X—Y is CH═N— and a physiologically-hydrolyzable and -acceptable ether or ester thereof when R₅ is hydroxy, in free form or in salt form (referred to herein as “compounds of formula I”)

In an even more preferred embodiment the first agent is tegaserod. Tegaserod is a compound of formula

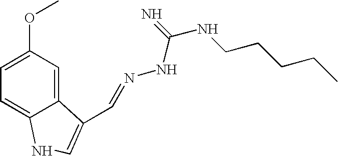

and is a partial agonist at serotonin type-4 (5-HT₄) receptors. Tegaserod is structurally derived from the physiological ligand of 5-HT₄ receptors, serotonin. The chemical name is 3-(5-Methoxy-1H-indol-3-ylmethylene)-N-pentylcarbazimidamide and its empirical formula is C₁₆H₂₃N₅O. The molecular weight is 301.39 and its chemical structure is set forth above. One skilled in the art can synthesize tegaserod by the teachings of U.S. Pat. No. 5,510,353 issued to Giger and from the teachings herein. Tegaserod is specifically described in example 13 of co-owned U.S. Pat. No. 5,510,353. Tegaserod may be in free form or in a pharmaceutically acceptable salt form. A preferred salt form is hydrogen maleate.

The co-agents which are combinable with the first agent include, but are not limited to, the following:

1) 5-HT₄ receptor antagonists which are compounds which bind to the 5-HT₄ receptor as defined by the IUPHAR (See Pharmacological Reviews, Vol. 44, p.157-213 (1994) and that antagonize the effect of serotonin. A relevant test to determine whether or not a compound is a 5-HT₄ receptor antagonist is the Guinea-Pig distal colon test as described in Br. J. Pharm., p. 1593-1599 (1993) or in the test described in Arch. Pharmacol., Vol. 343, p.439-446 (1991). Representative 5-HT₄ receptor antagonists include:

A-85380 (Abbott Laboratories) (WO 94/08994)

SB 204070 (SmithKline Beecham) (Drugs Fut., 19:1109-1121 (1994))

SB 207266 (piboserod) (SmithKline Beecham) (Marketletter, 22-1 22 en 22-18 (1995))

SB 207058 (SmithKline Beecham) (Exp. Opin. Invest. Drugs, 3(7):767 (1994))

SB 207710 (SmithKline Beecham) (Drug Data Report, 15(10):949 (1993))

SB 205800 (SmithKline Beecham) (Drug Data Report, 15(10):949 (1993))

SB 203186 (SmithKline Beecham) (Br. J. Pharmacol., 110:10231030 (1993))

N 3389 (Nisshin Flour Milling) (Eur. J. Pharmacol., 271:159 (1994))

FK 1052 (Fujisawa) (J. Pharmacol. Exp. Ther., 265:752 (1993))

SC 56184 (Searle) (R&D Focus, 2(37) 10 (1993))

SC 53606 (Searle/Monsanto) (J. Pharmacol. Exp. Ther. 226:1339)

DAU 6285 (Boerhinger Ingelheim) (Br. J. Pharmacol., 105;973 (1992))

GR 125487 (Glaxo) (Br. J. Pharmacol., 113 suppl. 119P & 120P (1994))

GR 113808 (Glaxo) (Br. J. Pharmacol. 110:1172)

RS 23597 (Syntex) (Bioorg Med. Chem. Lett., 4(20):2477 (1994))

RS 39604 (Syntex) (Br. J. Pharmacol., 115, 1087-1095 (1995))

RS 100235

LY0353433 (Eli Lilly co. Ltd.) (J. Pharmacol. Exp. Ther., 277(1), 97-104 (1996))

R 59595 (Eur. J. Pharmacol., 212 (1992), 51-59),

2) 5-HT₄ receptor agonists including but not limited to prucalopride; and mosapride. Mosapride is marketed in Japan under the tradename GASMOTIN®,

3) 5-HT₃ receptor antagonists which include compounds which bind to the 5-HT3 receptor and antagonize the effect of a 5-HT₃ receptor agonist, such as cilansetron; alosetron; ramosetron; azasetron; ondansetron; granisetron; and tropisetron,

4) compounds which exhibit characteristics of some or all of 1 through 3 above such as cisapride and nor-cisapride; BIMU compounds, for example BIMU1, BIMU8 and DAU 6215 (also known as itasetron) as disclosed in Dumuis A., et al., Naunyn Schmiedeber's Arch. Pharmacol., Vol. 343(3), pp. 245-251 (1991); DAU-6236 as disclosed in Rizzi, C. A. et al., J. Pharmacol. Exp. Ther., Vol. 261, pp. 412-419 (1992); and DAU-6258, Turconi M, et al., J. Med. Chem., Vol. 33(8), pg. 2101-2108 (1990), SDZ 205-557 which is a benzoic acid derivative (ester) Eglen R. M. et al., Proc. Br. Pharmacol. Soc., Vol. 149 (1992); renzapride; zacopride; SB 205149; SC 53116; RS 67333; RS 67506; and (S)-RS 56532, lintopride,

5) H₂ antagonists which include compounds which inhibit the action of histamine at the histamine H₂ receptors on gastric cells such as famotidine marketed under the trade name PEPCID®; cimetidine marketed under the trade name TAGAMET®; ranitidine marketed under the trade name ZANTAC®; and nizatidine marketed under the trade name AXID®,

6) Proton Pump Inhibitors (PPIs) including irreversible PPIs, which include compounds which inhibit gastric acid secretion by inhibition of H⁺/K⁺ ATPase enzyme system of the gastric parietal cells such as omeprazole marketed under the trade name PRILOSEC® and LOSEC®; lansoprazole marketed under the trade name PREVACID®; rabeprazole marketed under the trade name PARIET® and ACIPHEX®; pantroprazole marketed under the trade name PROTIUM®; and esomeprazole; reversible PPIs, which include, for example, BY 841, SKF 97574, SKF 96067, H 40502 and those disclosed in WO 98/43968 which are YH1238 and YH1885, Kim H. et al., Korean Journal of Physiology and Pharmacology, 1997, Vol 1(3), pp. 337-343; and pre-PPIs.

7) anxiolytics, for example, chlordiazepoxide,

8) benzodiazepine compounds and analogs which act to suppress seizures through an interaction with γ-aminobutyric acid (GABA) receptors of the A-type (GABA_{A}), for example, DIASTAT® and VALIUM®; LIBRIUM®; and ZANAX®,

9) anti-spasmodic/anti-muscarinic agents, for example, dicyclomine marketed under the trade name BENTYL®; hyoscyamine marketed under the trade name LEVSIN®; and darifenacin, (S)-1-[2-(2,3-Dihydro-5-benzofuranyl)ethyl]-α,α-diphenyl-3-pyrrolidineacetamide, described in U.S. Pat. No. 5,837,724 which is a selective muscarinic M₃ receptor antagonist,

10) SSRIs, for example, fluvoxamine; fluoxetine; paroxetine; sertraline; citalopram; venlafaxine; cericlamine; duloxetine; milnacipran; nefazodone; and cyanodothiepin (See The Year Drugs News, 1995 Edition, pp. 47-48 by Prous J. R.) and WO 97/29739,

11) tricyclic anti-depressants, for example, amitriptyline marketed under the trade names ELAVIL®, ETRAFON®, LIMBITROL®, and TRIAVIL®; buproprion; and Sinequan,

12) selegeline marketed under the trade names ELDEPRYL®, ATAPRYL® and DEPRENYL®,

13) belladonna alkaloids, for example, atropine and scopolamine,

14) M₁ antagonists,

15) metoclopramide marketed under the trade names REGLAN®,

16) CCK receptor antagonists, for example, devasepide; lorglumide; dexloxiglumide; loxiglumide, D'Amato, M. et al., Br. J. Pharmacol. Vol. 102(2), pp. 391-395 (1991); CI 988; L364,718; L363,260; L740,093 and LY288,513; and additional CCK receptor antagonists disclosed in U.S. Pat. No. 5,220,017, Bruley-Des-Varannes, S, et al. Gastroenterol. Clin. Biol. Vol. 15:(10), pp. 744-757 (1991), and Worker C: EPHAR '99—Second European Congress of Pharmacology (Part IV) Budapest, Hungary Iddb Meeting Report Jul. 3-7, 1999,

17) kappa opioid agonists or antagonists, for example, fedotozine,

18) motilin receptor agonists or antagonists, for example, motilin agonist ABT-269, (erythromycin, 8,9-didehydro-N-dimethyl-9-deoxo-4″,6,12-trideoxy-6,9-epoxy-N-ethyl), de(N-methyl-N-ethyl-8,9-anhydroerythromycin A and de(N-methyl)-N-isoprop-8,9-anhydroerythromycin A, Sunazika T. et al., Chem. Pharm. Bull., Vol. 37(10), pp. 2687-2700 (1989); A-173508 (Abbot Laboratories); motilin antagonists (Phe3, Leu13) porcine motilin, 214^{th} American Chemical Society (ACS) Meeting (Part V); Highlights from Medicinal Chemistry Poster Session, Wednesday 10 September, Las Vegas, Nevada, (1997), Iddb Meeting Report Se. 7-11, 1997; and ANQ-11125, Peeters T. L., et al., Biochem. Biophys. Res. Commun., Vol. 198(2), pp. 411-416 (1994),

19) nitric oxide synthase inhibitors,

20) GABA_{B} receptor agonists or modulators, for example, (±)-baclofen, S(−)-baclofen, R(+)-baclofen, CGP44532, CGP47656, CGP7930, SK&F97541

21) substance P agonists or antagonists,

22) NK receptor agonists or antagonists. Examples of NK receptor antagonists include FK 888(Fujisawa); GR 205171 (Glaxo Wellcome); LY 303870 (Lilly); MK 869 (Merck); GR82334 (Glaxo Wellcome); L758298 (Merck); L 733060 (Merck); L 741671 (Merck); L 742694 (Merck); PD 154075 (Parke-Davis); S18523 (Servier); S19752 (Servier); OT 7100 (Otsuka); WIN 51708 (Sterling Winthrop); NKP-608A; TKA457; DNK333; CP-96345; CP-99994; CP122721; L-733060; L-741671; L-742694; L-758298; L-754030; GR-203040; GR-205171; RP-67580; RPR-100893 (dapitant); RPR-107880; RPR-111905; FK-888; SDZ-NKT-343; MEN-10930; MEN-11149; S-18523; S-19752; PD-154075 (CAM-4261); SR-140333; LY-303870 (lanepitant); EP-00652218; EP-00585913; L-737488; CGP-49823; WIN-51708; SR-48968 (saredutant); SR-144190; YM-383336; ZD-7944; MEN-10627; GR-159897; RPR-106145; PD-147714 (CAM-2291); ZM-253270; FK-224; MDL-105212A; MDL-105172A; L-743986; L-743986 analogs; S-16474; SR-142801 (osanetant); PD-161182; SB-223412; and SB-222200,

23) calcitonin gene-related peptide (CGRP) receptor agonists or antagonists, which includes CGRP-(8-37), Onodera S, et al., Jpn. J. Pharmacol., Vol. 68(2), pg. 161-173 (1995) and Daines R. A. et al., Bioorganic Med. Chem. Lett., Vol. 7(20), pg. 2673-2676 (1997),

24) Endorphin/Enkephalin analogs,

25) anti-inflammatory compounds, particularly those of the immunomodulatory type, for example, NSAIDS; immunomodulatory drugs; Tumor Necrosis Factor (TNF, TNFα) inhibitors; basiliximab (e.g. SIMULECT®); daclizumab (e.g. ZENAPAX®); infliximab (e.g. REMICADE®); mycophenolate mofetil (e.g. CELLCEPT®); azathioprine (e.g. IMURAN®); tacrolimus (e.g. PROGRAF®); steroids; and GI anti-inflammatory agents, for example, sulfasalazine (e.g. AZULFIDINE®); olsalazine (e.g. DIPENTUM®); and mesalamine (e.g. ASACOL®, PENTASA®, ROWASA®),

26) stimulant laxatives, for example, bisacodyl marketed under the trade names DULCOLAX®, FLEET® and EVAC-Q-KWIK®; and the non-prescription EX-LAX® (Novartis Consumer Health Inc.),

27) osmotic laxatives, for example, activated charcoal with sorbitol marketed under the trade name ACTIDOSE with SORBITOL®; and phosphate buffered saline,

28) fecal softeners, for example, senna concentrate marketed under the trade names X-PREP® and SENEKOT®,

29) absorbents and fiber supplements including bulk fiber laxative plus natural, vegetable stimulant marketed under the trade name PERDIEM®; and bulk forming natural therapeutic fiber, for example, METAMUCIL® and FIBERCON®,

30) antacids, such as aluminum and magnesium antacids; and calcium hydroxides such as MAALOX®,

31) GI relaxants, for example, cholestyramine resin marketed under the trade name LoCHOLEST® and QUESTRAN®,

32) loperamide, an anti-diarrhea agent, for example, IMODIUM®,

33) diphenoxylate, ethyl 1-(3-cyano-3,3-dipheny;propyl)-4-phenylisonipecotate, for example, LOMOTIL®,

34) anti-gas compounds, for example, simethicone marketed under the trade names MYLANTA® and MYLICON®; and enzyme preps including PHAZYME® and BEANO®

35) bismuth-containing preparations, for example, bismuth subsalicylate also known as PEPTO-BISMOL®,

36) dextromethorphans, for example, dextromethorphan HBr, 3methoxy-17-methyl-9α, 13α, 14α-morphinan hydrobromide monohydrate, trade names under which dextromethorphans are marketed include BROMFED-DM®, DIABE-TUSS DM®, and TYLENOL®,

37) pentosan polysulfate, a heparin-like macromolecular carbohydrate derivative which chemically and structurally resembles glycosaminoglycans, marketed under the trade name ELMIRON®,

38) hydroxyzine, for example hydroxyzine HCl, 1-(p-chlorobenzhydryl) 4-[2-(2-hydroxyethoxy)-ethyl] piperizine dihydrochloride, marketed under the trade name ATARA®,

39) mast cell stabilizers, for example, ketotifen marketed under the trade name ZADITEN®, and

40) anti-emetic dopamine D₂ antagonists, such as domperidone.

Also included are the pharmaceutically acceptable salts, racemates or enantiomers of all the above-identified co-agents.

Also within the scope of this invention is the combination of more than two separate active ingredients as set forth above, i.e. a pharmaceutical combination within the scope of this invention could include three active ingredients or more. Further, both the first agent and the co-agent are not the identical active ingredient.

In accordance with another aspect of the invention there is provided a pharmaceutical composition comprising a combination of a first agent and a co-agent as active ingredients, or pharmaceutically acceptable salts, racematesor enantiomers thereof, in the presence of a pharmaceutically acceptable carrier, and optionally, other therapeutic ingredients. In a preferred embodiment the first agent is a 5-HT₄ receptor partial agonist, for example, the compounds of formula I of co-owned U.S. Pat. No. 5,510,353. In an even more preferred embodiment the first agent is tegaserod.

Applicants have surprisingly found that the pharmaceutical combinations and compositions of the present invention provide an enhanced treatment response for the gastrointestinal altered motility, sensitivity and secretion and abdominal viscera disorders mentioned herein. For example, the combination of Tegaserod and PPIs provide not only motility regulation but inhibit gastric acid secretion as well which is extremely beneficial for GERD. Applicants have also surprisingly found that the pharmaceutical combinations and compositions of the present invention provide an enhanced reduction of gastrointestinal pain normally associated with the gastrointestinal altered motility, sensitivity and secretion and abdominal viscera disorders.

The term “pharmaceutically acceptable salts” or “a pharmaceutically acceptable salt thereof” refer to salts prepared from pharmaceutically acceptable nontoxic acids or bases including inorganic acids and bases. Suitable pharmaceutically acceptable acid addition salts for the first agent and the co-agents of the present invention include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like.

To prepare the pharmaceutical compositions of the present invention, the first agent and a co-agent, or their pharmaceutically acceptable salts, racematesor enantiomers are combined in intimate admixture by mixing, blending or combining in any manner known to those of skill in the art, with a pharmaceutically acceptable carrier. The pharmaceutically acceptable carrier may take a wide variety of forms depending on the form of preparation desired for administration.

Any suitable route of administration may be employed for providing a mammal with a therapeutically effective amount of the pharmaceutical combinations and compositions of the present invention. For example, oral, rectal, vaginal, topical, parental (subcutaneous, intramuscular, intravenous, transdermal) and like forms of administration may be employed. Dosage formulations include ointments, foams, gels, transdermal patches, tablets (both fractionable and non-fractionable), caplets, powders for inhalations, gelcaps, capsules, elixirs, syrups, chewable tablets, lozenges, troches, dispersions, aerosols, solutions, fast-dissolving wafers, suppositories or suspensions or other known and effective delivery methods.

In addition to the dosage formulations set out above, the pharmaceutical combinations and compositions of the present invention may also be administered by controlled release means and/or delivery devices such as those described in U.S. Pat. Nos. 3,845,770; 3,916,899; 3,536,809; 3,598,123; and 4,008,719 and by “fast-melt” means which include delivery devices which rapidly dissolve in the mouth. Rapid dissolution is meant to include dissolution which takes place in the patient's mouth within less than three minutes. Delivery devices for this type of formulation include, but are not limited to, tablets and capsules. An example of a fast-melt means as used herein is described in U.S. Pat. No. 5,178,878 which discloses an effervescent dosage form with microparticles for rapid dissolution of the tablet or capsule.

Oral dosing is preferred. In preparing the compositions in oral dose form, any of the usual pharmaceutical carriers may be employed including any material, composition or vehicle, such as a liquid or solid filler, diluent, excipient, solvent or encapsulating material involved in carrying, formulating or transporting a chemical agent. Specific examples are water, glycols, oils, alcohols and the like in the case of oral liquid preparations. In oral solid forms solid carriers such as starches, sugars, kaolin, lubricants, binders, disintegrating agents and the like may be employed. Oral solid preparations are preferred over the oral liquid preparations. A preferred oral solid preparation is capsules and tablets, because of their ease of administration.

For parental compositions, the carrier will usually comprise sterile water, at least in large part, though other ingredients, to aid solubility for example, may be included. Injectable solutions, for example, may be prepared in which the carrier comprises PEG, saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wetting agent, optionally combined with suitable additives of any nature in minor proportions, which additives do not cause a significant deleterious effect on the skin. It is especially advantageous to formulate the aforementioned pharmaceutical compositions in dosage unit form for ease of administration and uniformity of dosage. Dosage unit form as used herein refers to physically discrete units suitable as unitary dosages, each unit containing a predetermined quantity of active ingredient(s) calculated to produce the desired therapeutic effect in association with the required pharmaceutical carrier.

In another embodiment of the present invention there is provided a method of treating a patient suffering from a gastrointestinal altered motility, sensitivity and secretion and abdominal viscera disorder comprising administering a therapeutically effective amount of a pharmaceutical composition comprising a combination of a first agent and a co-agent, or the pharmaceutically acceptable salts, racematesor enantiomers thereof, in the presence of a pharmaceutically acceptable carrier to the patient. In a preferred embodiment the first agent is the compounds of formula I of co-owned U.S. Pat. No. 5,510,353. In an even more preferred embodiment the first agent is tegaserod.

Generally, the pharmaceutical compositions of the present invention are employed for the treatment of a gastrointestinal altered motility, sensitivity and secretion and abdominal viscera disorder including, but not limited to, heartburn, bloating, postoperative ileus, abdominal pain and discomfort, early satiety, epigastric pain, nausea, vommitting, regurgiation, burbulence, intestinal pseudoobstruction, anal incontinence, GERD, IBS, dyspepsia, chronic constipation, diabetic gastroparesis, ulcerative colitis, Crohn's disease, menstrual cramps, spastic and interstitial cystitis and ulcers and the visceral pain associated therewith. In addition, the pharmaceutical combinations and compositions may also be employed as laxatives, as a preparation for a patient for colonoscopy, and forregulating and stabilizing enterochromaffin cell secretory, pain and motility mechanisms, afferent fiber activity and GI and lower abdominal smooth muscle cells.

More specifically, the first agent(s) and the co-agents described above may be combined as follows:

5-HT₄ receptor agonists or antagonists or 5-HT₃ antagonists to treat dyspepsia, GERD, IBS and abdominal viscera disorders;

H₂ antagonists or PPIs to treat dyspepsia, GERD, IBS and abdominal viscera disorders;

compounds from category 4) above (compounds which exhibit mixed 5-HT₃ and 5-HT₄ receptor modulating properties), cilansetron, darifenacin or piboserod to treat IBS, GERD, dyspepsia and abdominal viscera disorders;

anxiolytics, anti-spasmodic/anti-muscarinic agents, buproprion, belladonna alkaloids, endorphin/enkephalin analogs or GI anti-inflammatory agents to treat IBS, GERD, dyspepsia and abdominal viscera disorders;

SSRIs, M₁ antagonists, metoclopramide, CCK antagonists or motilin receptor agonists or antagonists to treat functional dyspepsia and IBS, GERD and abdominal viscera disorders;

pentosan polysulfate or hydroxyzine to treat cystitis, IBS, GERD, dyspepsia and abdominal viscera disorders;

tricyclic antidepressants, for example, amitriptyline to treat IBS, GERD, dyspepsia and abdominal viscera disorders; and

anti-gas compounds to treat dyspepsia, IBS, GERD and abdominal visceral pain. The therapeutically effective dosage of the pharmaceutical compositions of this invention will vary with the severity of the condition to be treated, and the route of administration. The dose, and perhaps the dose frequency, will also vary according to the age, body weight, and response of the individual patient. In general, the combination of the first agent and the co-agent may be administered in a molar ratio having a range of from about 0.01 to about 2 for the first agent to a range of from about 0.01 to 1000 for the co-agent. As an example, the molar ratio for the first agent to the co-agent is about 1:1000 (first agent to co-agent). As a more specific example, the molar ratio for the first agent to the co-agent may be about 1:1000, 1:500, 1:200, 1:100, 1:20, 1:5, 1:1 or 1:0.01. A preferable molar ratio is about 1:20, even more preferably about 1:5 and most preferably about 1:1.

The total daily dose range, which comprises the above-described molar ratio, for the conditions described herein, may be administered in a range of from about 0.01 mg to about 1000 mg. The daily dose range may be about 800 mg, 600 mg, 400 mg, 200 mg, 100 mg, 50 mg, 20 mg, 10 mg, 5 mg, 1 mg, 0.1 mg or 0.01 mg. Preferably, a daily dose range should be between about 0.5 mg to about 100 mg, while most preferably, a daily dose range should be between about 5 mg to about 75 mg. It is preferred that the doses are administered OD (once daily) or BID (2 times a day). In managing the patient, the therapy should be initiated at a lower dose, perhaps about 5 mg to about 10 mg, and increased up to about 50 mg or higher depending on the patient's response. It may be necessary to use dosages outside these ranges in some cases as will be apparent to those skilled in the art. Further, it is noted that the clinician or treating physician will know how and when to interrupt, adjust, or terminate therapy in conjunction with individual patient response. The term “therapeutically effective amount” is encompassed by the above-described molar ratio and dosage amounts and dose frequency schedule.

### EXAMPLES

The present invention is further described by the following examples. The examples are provided solely to illustrate the invention by reference to specific embodiments. These exemplification's, while illustrating certain specific aspects of the invention, do not portray the limitations or circumscribe the scope of the disclosed invention.

### Example 1

### Treatment of Non-Erosive GERD with the Combination of Tegaserod and a Co-Agent

Patients selected for the study are patients with heartburn, the target symptom in patients with non-erosive GERD, as the predominant upper gastrointestinal symptom during the last three (3) months prior to entry into the study and with a history of episodes of heartburn occurring on at least 3 days/week. Patients with GERD and without endoscopic signs of erosive esophagitis are included in the study. Among other factors, patients who are treated with H₂RAs in prescription doses or PPIs within one month prior to entry into the baseline phase of the study (Day −14) and patients who need continuous use of PPIs within three months prior to entry into the baseline phase of the study are excluded.

The study consists of a one-week screening period and a 2-week drug-free baseline period, followed by an 8-week, double-blind, placebo-controlled treatment period. During the screening period (Day −21 to Day −14), an endoscopy is performed to rule out the presence of erosive esophagitis. During baseline (Day −14 to Day 1), the patient's symptoms of GERD are documented in a daily diary. At the start of the period, medications for GERD such as histamine H₂-receptor antagonists (H₂RAs), proton pump inhibitors (PPIs), prokinetics and other disallowed medication are withdrawn and the patients are instructed not to change their diet or lifestyle during the trial. Patients are allowed to take Maalox tablets as a rescue medication for the control of their symptoms. Patients entering the double-blind period have episodes of heartburn on three (3) or more days the last week of the baseline period. Patients are randomized in equal groups during the double-blind, placebo-controlled period of the study. This period of the study lasts eight (8) weeks and there are 12 treatment arms. The patients in each group receive one of the following regimens: 1) placebo, 2) tegaserod 0.4 mg/day, 3) tegaserod 1 mg/day, 4) tegaserod 4 mg/day, 5) ranitidine 300 mg/day, 6) omeprazole 20 mg/day, 7) tegaserod 0.4 mg/day plus ranitidine 300 mg/day, 8) tegaserod 1 mg/day plus ranitidine 300 mg/day, 9) tegaserod 4 mg/day plus ranitidine 300 mg/day, 10) tegaserod 0.4 mg/day plus omeprazole 20 mg/day, 11) tegaserod 1 mg/day plus omeprazole 20 mg/day and 12) tegaserod 4 mg/day plus omeprazole 20 mg/day, given orally bid for the eight (8) weeks. The administration as per the twelve (12) groups above is within thirty (30) minutes before meal time in the morning and in the evening. During the eight (8) weeks, patients continue to complete the daily diary and use only Maalox tablets as rescue medication for the control of their symptoms.

The combination of 1) tegaserod with omeprazole and 2) tegaserod with ranitidine significantly reduces the episodes of heartburn occurring per week during the eight (8) week period of the double-blind, placebo-controlled period of the study as compared to any of placebo, tegaserod, ranitidine and omeprazole alone. The tegaserod combinations also reduces the other symptoms of GERD including, abdominal pain, bloating and regurgitation. Further, patients show a significant improvement in quality of life factors as compared to any of placebo, tegaserod, ranitidine and omeprazole alone.

### Example 2

### Pharmacodynamic Effects of Tegaserod and Co-agents on Gastrointestinal and Colonic Motility

Animal preparation: Beagle dogs are used in these experiments. Under halothane anesthesia, four strain-gauge transducers constructed according to Pascaud et al. (Am. J. Physiol. (1978) 235: E532-E538) are sewn on the serosa of the antrum at 5 cm from the pylorus, the duodenum at 10 cm from the pylorus, the jejunum at 50 cm from the ligament of Treitz and the proximal colon at 10 cm from the ileo-colonic junction. Each transducer is sewn with its recording axis parallel to the transverse axis of the gut to measure the contractile force of the circular muscle layer. The free ends of the strain-gauge wires are drawn sub-cutaneously to emerge dorsally between the scapulas.

Recordings: Calibration of each strain-gauge is performed before implantation. Mechanical activity detected by the transducers is recorded. The motility index of the antrum, duodenum, jejunum and colon is determined according to the technique of Hachet et al. (J. Pharmacol. Meth. (1986) 16: 171-180). The calculated index of motility corresponded to the area between the baseline and the contractile curve during 30 min intervals.

Study design: The dogs were separated into groups. Each group receives one of the following regimens: 1) placebo, 2) tegaserod, 3) alosetron, 4) prucalopride, 5) A-85380, 6) fedotozine, 7) chlordiazepoxide, 8) CGP-49823, 9) buproprion, 10) fluvoxamine, 11) tegaserod plus alosetron, 12) tegaserod plus prucalopride, 13) tegaserod plus A-85380, 14) tegaserod plus fedotozine, 15) tegaserod plus chlordiazepoxide, 16) tegaserod plus CGP-49823, 17) tegaserod plus buproprion, or 18) tegaserod plus fluvoxamine. Compounds at different doses or placebo are administered p.o. to fasted dogs 30 min prior to a meal (water ad libitum). Intravenous infusions of compounds at different doses or placebo (vehicle) to fasted dogs started 30 min prior to a meal (water ad libitum). Gastrointestinal and colonic motility recordings started with the meal intake and are carried out for 6 hours duration in total.

Data analysis: Changes in motility index during the 6 hours after the meal associated with the different compounds/administrations are determined at the level of the antrum, duodenum, jejunum and colon.

The combination of tegaserod plus a co-agent significantly increased gastrointestinal and colonic motility as compared to placebo and any of the compounds administered alone.

### Example 3

### Effects of Tegaserod and Co-agents on Gastric and Colonic Sensitivity to Distension and on the Muscular Tone of the Gut Using Barostatic Distension

Gastric Sensitivity and Tone

Groups of Wistar rats weighing 200-250 g are used. For surgery, the animals are premedicated with 0.3 ml of acepromazine (0.5 mg/kg) injected intraperitoneally (ip) and anesthetized with 0.3 ml of ketamine injected intraperitoneally. Animals are positioned in dorsal decubitus and following a xypho-ombilical laparotomy, the stomach is fitted with a permanent balloon connected to a tube introduced in the upper part of the rumen at 1 cm of the gastro-esophageal junction on the great curvature. After closure of the abdomen, rats are positioned in ventral decubitus and one group of 3 stainless steel electrodes (1 m in length-270 μm in diameter) is implanted into the neck muscles using a technique described in (Ruckebusch and Fioramonti, 1975). The free ends of electrodes and the catheter of the balloon are exteriorized on the back of the neck and protected by a glass tube attached to the skin.

Gastric distension at constant pressure is performed with an electronic barostat (Hachet et al., Gastroenterol Clin Biol, 1993, 17, 347-351). Balloons (5.0-5.5 cm in length) are made with cistern free condoms and sutured to a polyethylene tube (1.0 and 1.8 mm inner and outer diameter respectively, 80 cm in length). The end of the tube is drilled for an easier emptying of the balloon.

Ten days after surgery, electromyographic recordings are performed with an electroencephalograph machine (Reega VIII, Alvar, Paris, France) at a paper speed of 2.4 cm/min. A short time constant of amplification is used to record selectively spike burst (0.03 s). The electromyographic activity is summed every 20 s by an integrator circuit and automatically plotted on a computer.

Under noxious gastric distension, the rat stretches its body and rises up the head and/or turns the head on the left and right sides to observe his flank. The neck muscles are contracted and an electromyographic signal is recorded. In addition, the barostat is connected to a potentiometric recorder for the permanent recording of intragastric pressure. The animals are separated into groups.

After a 30 min period of control recording, the animals receive one of the following regimens: 1) placebo, 2) tegaserod, 3) ranitidine, 4) omeprazole, 5) alosetron, 6) prucalopride, 7) A-85380, 8) fedotozine, 9) chlordiazepoxide, 10) CGP49823, 11) buproprion, 12) fluvoxamine, 13) tegaserod plus alosetron, 14) tegaserod plus prucalopride, 15) tegaserod plus A-85380, 16) tegaserod plus fedotozine, 17) tegaserod plus chlordiazepoxide, 18) tegaserod plus CGP-49823, 19) tegaserod plus buproprion, 20) tegaserod plus fluvoxamine, 21) tegaserod plus ranitidine, or 22) tegaserod plus omeprazole.

The protocol of gastric distension is started 30 min later.

Electromyographic activity of the neck muscles (EANM) is correlated with changes of posture and is proportional to pain induced by gastric distension. Values integrated every 20 s are summed up for consecutive 10 min. For each stage of distension, neck activity is determined with the following formula:

$$\frac{\begin{matrix}( EANM at a determined pressure ) - \\ ( EANM in basal conditions )\end{matrix}}{EANM in basal conditions} * 100$$

The pain threshold is determined as an increase >100 % of the electrical activity of the neck muscles.

Gastric volume is determined on the potentiometric recorder as the maximal volume obtained for each stage of distension. Pain threshold and gastric volume are given as mean±SEM and values compared using Student's “t” test for unpaired values.

The pharmaceutical combination of tegaserod and a co-agent significantly decreased the gastric pain associated with gastric distension and increased gastric tone as compared to placebo and any of the compounds administered alone.

Colorectal Sensitivity and Tone

The influence of tegaserod and a co-agent on rectal or colonic tone and pain is done using barostat distension procedures by applying increasing pressure in a stair-case manner for consecutive periods of 5 min.; the volume is measured for each pressure giving an evaluation of the changes in tone.

Wistar rats weighing 220-250 g and housed individually are used. The animals are premedicated with 0.5 mg/kg of acepromazine injected intraperitoneally (IP) and anesthetized by intramuscular administration of 100 mg/kg of ketamine. They are prepared for electromyographic recordings using the technique described in (Ruckebusch and Fioramonti, 1975). Pairs of nichrome wire electrodes (60 cm in length and 80 μm in diameter) are implanted in the striated muscle of the abdomen, 2 cm laterally from the white line. The free ends of electrodes are exteriorized on the back of the neck and protected by a plastic tube attached to the skin.

Electromyographic recordings (time constant: 0.03 sec) started 8 days after surgery. Bipolar recordings of myoelectric activity are performed with an electroencephalographic recorder during one hour starting 30 min before rectal distension.

In order to prevent recording artefacts due to movements during distension, rats are acclimated, 3 days before distension, to stay in tunnel of polypropylene in which distension and EMG recordings are performed. A balloon consisting of a condom (4 cm) is introduced into the rectum at 5 cm from the anus and fixed at the base of the tail. The balloon, connected to a barostat, is increasingly inflated with air at pressures of 15, 30, 45 and 60 mmHg. each pressure being applied during 5 min.

Groups of rats are submitted respectively to the barostatic distension protocol. Ten minutes before they are injected IP with 1) placebo, 2) tegaserod, 3) ranitidine, 4) omeprazole, 5) alosetron, 6) prucalopride, 7) A-85380, 8) fedotozine, 9) chlordiazepoxide, 10) CGP49823, 11) buproprion, 12) fluvoxamine, 13) tegaserod plus alosetron, 14) tegaserod plus prucalopride, 15) tegaserod plus A-85380, 16) tegaserod plus fedotozine, 17) tegaserod plus chlordiazepoxide, 18) tegaserod plus CGP-49823, 19) tegaserod plus buproprion, 20) tegaserod plus fluvoxamine, 21) tegaserod plus ranitidine, 22) tegaserod plus omeprazole.

Statistical analysis of the number of abdominal spike bursts occurring during each 5 min period are performed by Student's “t” test fair paired values comparisons after two way ANOVA. P<0.05 is considered statistically significant. Colorectal volumes are given as mean±SEM and values compared using Student's “t” test for unpaired values.

The pharmaceutical combination of tegaserod plus a co-agent significantly decreased the rectal and colonic pain associated with rectal distension and increased colorectal tone as compared to placebo and any of the compounds administered alone.

### Example 4

### Treatment of GERD with Tegaserod and a Co-Agent

This example is to determine the effect of variations in intrabolus pressure on esophageal peristalsis. In cats, intrabolus pressure is altered by increasing intragastric pressure to 20-45 mmHg by use of a pressure cuff to compress the abdomen. In each cat, increases in intragastric pressure are associated with comparable increases in pressure of the esophageal bolus while the bolus is in the distal esophagus during esophageal peristalsis. Secondary peristalsis induced by a 5-ml injection of barium into the proximal esophagus is recorded by synchronized video fluoroscopy and esophageal manometry. Graded increases in intrabolus pressure caused an increased prevalence of ineffective, incomplete peristaltic sequences that did not completely clear barium from the esophagus. At intragastric pressures greater than 45 mmHg, 63% of the peristaltic sequences are incomplete. Increases in intrabolus pressure elicited by increased intragastric pressure also caused 1) slowing of the peristaltic wave in the distal esophagus, 2) increased pressure wave duration in the distal esophagus, 3) increased esophageal diameter, and 4) increased duration of lower esophageal sphincter opening. The incidence of retrograde bolus escape is inversely related to the difference between peristaltic wave amplitude and intrabolus pressure. A pressure difference of greater than 20 mmHg prevented retrograde barium escape at all esophageal levels, whereas a difference of less than 20 mmHg is generally associated with retrograde escape of barium in the distal esophagus.

The cats are randomized in equal groups with each group receiving one of the following regimens: 1) placebo, 2) tegaserod 3) granisetron 4) loxiglumide 5) fedotozine 6) motilin 7) tegaserod plus granisetron, 8) tegaserod plus loxiglumide, 9) tegaserod plus fedotozine, and 6) tegaserod plus motilin.

In the placebo group, an increase in intrabolus pressure causes an increase in esophageal distension while in the drug treated groups the esophageal distension is decreased.

### Example 5

### The Effect of Tegaserod and a Co-Agent on the Release of Serotonin (5HT) from Guinea-Pig Colon Entero-Chromaffin Cells

Male guinea-pigs, 200-400 g are stunned and bled. Segments of the proximal colon are removed and suspended, as described by Trendelenburg in Arch. Exp. Path. Pharmakol., 81, 55-129 (1917), in a 20 ml organ bath. The tissue is bathed with a modified Krebs solution (NaCl 118.6; CaCl.sub.2 2.7; KCl 4.7; KH.sub.2 PO.sub.4 1.2; MgSO.sub.4 0.1; NaHCO.sub.3 25.0; and glucose 5.6 mM), maintained at 37° C. 5-HT is determined by high-performance liquid chromatography with electro-chemical detection. The luminal outflow of 5-HT is significantly reduced by atropine (0.2 microM) and hexamethonium (100 microM). Physostigmine (1 microM) causes a great increase (atropine-sensitive) in 5-HT outflow from entero-chromaffin cells. To individual organ baths the following agents are added prior to the addition of physostigmine (1 microM): 1) no addition (negative control), 2) atropine (0.2 microM) (positive control), 3) hexamethonium (100 microM) (positive control), 4) tegaserod, 5) cilansetron, 6) prucalopride, 7) A-85380, 8) fedotozine, 9) chlordiazepoxide, 10) CGP49823, 11) buproprion, 12) fluvoxamine, 13) tegaserod plus cilansetron, 14) tegaserod plus prucalopride, 15) tegaserod plus A-85380, 16) tegaserod plus fedotozine, 17) tegaserod plus chlordiazepoxide, 18) tegaserod plus SDZ-NKT-343, 19) tegaserod plus buproprion, or 20) tegaserod plus fluvoxamine. The combination of agents results in an amplification of the reduction of physostigmine-induced serotonin outflow as compared to any of the agents alone.

Although the present invention has been described in considerable detail with reference to certain preferred versions thereof, other versions are possible without departing from the spirit and scope of the preferred versions contained herein. All references and Patents (U.S. and others) referred to herein are hereby incorporated by reference in their entirety as if set forth herein in full.

What is claimed is:

1. A pharmaceutical combination comprising:
a) a first agent selected from the group consisting of 5-HT₄ receptor agonists or antagonists, 5-HT₄ receptor partial agonists and 5-HT₃ receptor antagonists; and
b) a co-agent.

2. The pharmaceutical combination of claim 1 wherein the first agent and the co-agent include pharmaceutically acceptable salts, racemates or enantiomers thereof.

3. The pharmaceutical combination of claim 1 wherein the first agent is selected from the group consisting of compounds of formula I, tegaserod, cisapride, nor-cisapride, renzapride, zacopride, mosapride, prucalopride, SB 205149, SC 53116, RS 67333, RS 67506, BIMU 1, BIMU 8 and (S)-RS 56532.

4. The pharmaceutical combination of claim 1 wherein the first agent is tegaserod.

5. The pharmaceutical combination of claim 1 wherein the first agent is the compounds of formula I.

6. A pharmaceutical composition comprising the pharmaceutical combination of claim 2 and a pharmaceutically acceptable carrier.

7. The pharmaceutical composition of claim 6 wherein the co-agent is selected from the group consisting of 5-HT₃ receptor antagonists, 5-HT₄ receptor agonists or antagonists, compounds which show characteristics of 5-HT₃ receptor antagonists and 5-HT₄ receptor agonists or antagonists, H₂ antagonists, PPIs, anxiolytics, benzodiazepine compounds, antispasmodic/anti-muscarinic agents, SSRIs, tricyclic antidepressants, selegeline, belladonna alkaloids, M₁ antagonists, metoclopramide, CCK receptor antagonists, kappa opioid agonists or antagonists, motilin receptor agonists or antagonists, nitric oxide synthase inhibitors, BIMU compounds, GABA_{B} receptor agonists or modulators, NK receptor agonists or antagonists, substance P agonists or antagonists, calcitonin gene-related peptide receptor agonists or antagonists, endorphin/enkephalin analogs, anti-inflammatory compounds, stimulant laxatives, osmotic laxatives, fecal softeners, absorbents and fiber supplements, antacids, GI relaxants, loperamide, diphenoxylate, anti-gas compounds, bismuth-containing preparations, subsalicylate, pentosan polysulfate, hydroxyzine, dextromethorphans, mast cell stabilizers and anti-emetic dopamine D₂ antagonists.

8. The pharmaceutical composition of claim 7 wherein the 5-HT₄ receptor antagonists are selected from the group consisting of A-85380, SB 204070, SB 207266, SB 207058, SB 207710, SB 205800, SB 203186, N 3389, FK 1052, SC 56184, SC 53606, DAU 6285, GR 125487, GR 113808, RS 23597, RS 39604, RS 100235, LY0353433 and R 59595.

9. The pharmaceutical composition of claim 7 wherein the 5-HT₄ receptor agonists are selected from the group consisting of mosapride and prucalopride.

10. The pharmaceutical composition of claim 7 wherein the 5-HT₃ receptor antagonists are selected from the group consisting of cilansetron, ramosetron, azasetron, ondansetron, granisetron, tropisetron and alosetron.

11. The pharmaceutical composition of claim 7 wherein the compounds which show characteristics of 5-HT₃ receptor antagonists and 5-HT₄ receptor agonists or antagonists are selected from the group consisting of cisapride, nor-cisapride, SDZ 205-557 and BIMU compounds.

12. The pharmaceutical composition of claim 11 wherein the BIMU compounds are selected from the group consisting of BIMU1, BIMU8, DAU 6215, and DAU-6258.

13. The pharmaceutical composition of claim 7 wherein the H₂ antagonists are selected from the group consisting of famotidine, cimetidine, nizatidine and ranitidine.

14. The pharmaceutical composition of claim 7 wherein the PPIs are irreversible PPIs selected from the group consisting of omeprazole, rabeprazole, pantoprazole, esomeprazole and lansoprazole.

15. The pharmaceutical composition of claim 7 wherein the PPIs are reversible PPIs selected from the group consisting of BY 841, SKF 97574, SKF 96067, H 40502, BY 112, YH1238 and YH1885.

16. The pharmaceutical composition of claim 7 wherein the PPIs are pre PPIs.

17. The pharmaceutical composition of claim 7 wherein the anti-emetic dopamine D₂ antagonist is domperidone.

18. The pharmaceutical composition of claim 7 wherein the benzodiazepine compounds or analogs are selected from the group consisting of LIBRIUM®, ZANAX®, DIASTAT® and VALIUM®.

19. The pharmaceutical composition of claim 7 wherein the antispasmodic/anti-muscarinic agents are selected from the group consisting of dicyclomine, darifenacin and hyoscyamine.

20. The pharmaceutical composition of claim 7 wherein the SSRIs are selected from the group consisting of fluvoxamine, fluoxetine, paroxetine, sertraline, citalopram, venlafaxine, cericlamine, duloxetine, milnacipran, nefazodone, and cyanodothiepin.

21. The pharmaceutical composition of claim 7 wherein the tricyclic antidepressants are selected from the group consisting of amitriptyline, sinequan and buproprion.

22. The pharmaceutical composition of claim 7 wherein the belladonna alkaloids are selected from the group consisting of atropine and scopolamine.

23. The pharmaceutical composition of claim 7 wherein the CCK receptor antagonists are selected from the group consisting of devasepide, lorglumide, loxiglumide, dexioxiglumide, CI 988, L364,718, L363,260, L740,093 and LY288,513.

24. The pharmaceutical composition of claim 7 wherein the kappa opioid agonist or antagonist is fedotozine.

25. The pharmaceutical composition of claim 7 wherein the motilin receptor agonists or antagonists are selected from the group consisting of ABT-269, (erythromycin, 8,9-didehydro-N-dimethyl-9-deoxo-4″,6,12-trideoxy-6,9-epoxy-N-ethyl), de(N-methyl-N-ethyl-8,9-anhydroerythromycin A and de(N-methyl)-N-isoprop-8,9-anhydroerythromycin A, A-173508, (Phe3, Leu13) porcine motilin and ANQ-11125.

26. The pharmaceutical composition of claim 7 wherein the CGRP receptor antagonist is CGRP-(8-37).

27. The pharmaceutical composition of claim 7 wherein the anti-inflammatory compounds are selected from the group consisting of NSAIDS, immunomodulatory drugs, TNF inhibitors, basiliximab, daclizumab, infliximab, mycophenolate mofeil, azathioprine, tacrolimus, steroids, sulfasalazine, olsalazine and mesalamine.

28. The pharmaceutical composition of claim 7 wherein the stimulant laxatives are selected from the group consisting of bisacodyl and EX-LAX®.

29. The pharmaceutical composition of claim 7 wherein the osmotic laxatives are selected from the group consisting of sorbitol and phosphate buffered saline.

30. The pharmaceutical composition of claim 7 wherein the fecal softener is senna concentrate.

31. The pharmaceutical composition of claim 7 wherein the absorbents and fiber supplements are selected from the group consisting of bulk fiber laxative plus natural vegetable stimulants and bulk forming natural therapeutic fiber.

32. The pharmaceutical composition of claim 7 wherein the antacids are selected from the group consisting of aluminum antacids, magnesium antacids and calcium hydroxides.

33. The pharmaceutical composition of claim 7 wherein the GI relaxant is cholestyramine resin.

34. The pharmaceutical composition of claim 7 wherein the anti-gas compounds are selected from the group consisting of simethicone and enzyme preps.

35. The pharmaceutical composition of claim 7 wherein the bismuth containing preparations is bismuth subsalicylate.

36. The pharmaceutical composition of claim 7 wherein the GABA_{B} receptor agonists or modulators are selected from the group consisting of (±)-baclofen, S(−)-baclofen, R(+)-baclofen, CGP44532, CGP47656, CGP7930, SK&F97541.

37. The pharmaceutical composition of claim 7 wherein the NK antagonist compounds are selected from the group consisting of FK 888; GR 205171; LY 303870; MK 869; GR82334; L758298; L 733060; L 741671; L 742694; PD 154075; S18523; S19752; OT 7100; WIN 51708; NKP-608A, TKA457, DNK333, CP-96345, CP-99994, CP122721, L-733060, L-741671, L-742694, L-758298, L-754030, GR-203040, GR-205171, RP-67580, RPR-100893; RPR-107880, RPR-111905, FK-888, SDZ-NKT-343, MEN-10930, MEN-11149, S-18523, S-19752, PD-154075; SR-140333, LY-303870, EP00652218, EP-00585913, L-737488, CGP-49823, WIN-51708, SR-48968, SR-144190, YM-383336, ZD-7944, MEN-10627, GR-159897, RPR-106145, PD-147714, ZM-253270, FK-224, MDL-105212A, MDL-105172A, L-743986, L-743986 analogs, S-16474, SR-142801, PD-161182, SB-223412, and SB-222200.

38. A process for preparing the pharmaceutical composition of claim 6 comprising combining a therapeutically effective amount of a first agent and a co-agent, or the pharmaceutically effective salts, racemates or enantiomers thereof, with a pharmaceutically acceptable carrier.

39. The process of claim 38 wherein the first agent is tegaserod.

40. A method of treating a patient suffering from a gastrointestinal altered motility, sensitivity and secretion disorder comprising administering a therapeutically effective amount of the pharmaceutical composition of claim 6 to a patient in need thereof.

41. The method of claim 40 wherein the gastrointestinal altered motility, sensitivity and secretion disorder is selected from the group consisting of heartburn, bloating, abdominal pain and discomfort, early satiety, epigastric pain, nausea, vomiting, regurgitation, chronic constipation, diabetic gastroparesis, dyspepsia, gastro-esophageal reflux disease, irritable bowel syndrome, ulcerative colitis, Crohn's disease, spastic cystitis, interstitial cystitis, post-operative ileus, intestinal pseudoobstruction, anal incontinence and ulcers and visceral pain associated therewith.

42. A method of treating a patient suffering from an abdominal viscera disorder comprising administering a therapeutically effective amount of the pharmaceutical composition of claim 6 to a patient in need thereof.

43. The method of claim 42 wherein the abdominal viscera disorder is selected from the group consisting of those conditions treated by regulation, stabilization and normalization of enterochromaffin cell secretory, pain and motility mechanisms, afferent fiber actvity and GI and lower abdominal smooth muscle cells.

44. The method of claim 40 wherein the pharmaceutical composition is administered once or twice daily in oral dosage form.

45. The method of claim 44 wherein the pharmaceutical composition is administered in fast-melt dosage form.

46. A method of facilitating the evacuation of the small and large intestine comprising administering a therapeutically effective amount of the pharmaceutical composition of claim 6 to a patient in need thereof.

47. A method of preparing a patient for colonoscopy comprising administering a therapeutically effective amount of the pharmaceutical composition of claim 6 to the patient.

48. A method of regulating and stabilizing enterochromaffin cell secretory, pain and motility mechanisms comprising administering a therapeutically effective amount of the pharmaceutical composition of claim 6 to a patient in need thereof.

49. A method of regulating and stabilizing afferent fiber activity comprising administering a therapeutically effective amount of the pharmaceutical composition of claim 6 to a patient in need thereof.

50. A method of regulating, stabilizing and normalizing GI and lower abdominal smooth muscle cells comprising administering a therapeutically effective amount of the pharmaceutical composition of claim 6 to a patient in need thereof.

51. A method of treating GERD comprising administering the pharmaceutical composition of claim 6 wherein the co-agent is selected from the group consisting of 5-HT₃ receptor antagonists, 5-HT₄ receptor agonists or antagonists, H₂ antagonists, PPIs and anti-gas compounds.

52. A method of treating dyspepsia comprising administering the pharmaceutical composition of claim 6 wherein the co-agent is selected from the group consisting of 5-HT₃ receptor antagonists, 5-HT₄ receptor agonists or antagonists, H₂ antagonists and anti-gas compounds.

53. A method of treating IBS comprising administering the pharmaceutical composition of claim 6 wherein the co-agent is selected from the group consisting of 5-HT₃ receptor antagonists, 5-HT₄ receptor agonists or antagonists, BIMU compounds, anxiolytics, anti-spasmodic/anti-muscarinic agents, SSRIs, M₁ antagonists, metoclopramide, CCK antagonists, NK antagonists, motilin receptor agonists or antagonists, amitriptyline, buproprion, belladonna alkaloids, endorphin/enkephalin analogs and anti-inflammatory compounds.

54. The pharmaceutical composition of claim 7 wherein the mast cell stabilizer is ketotifen.
